(19) United States
(12) Patent Application Publication
(10) Pub. No.: US 2008/0082015 A1
(43) Pub. Date: Apr. 3, 2008

(54) PRE-CONDITIONED ECG SYSTEM AND METHOD

(76) Inventors: Mark Kohls, New Berlin, WI (US); Gordon Ian Rowlandson, Milwaukee, WI (US); Joel Q. Xue, Germantown, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/538,343

(22) Filed: Oct. 3, 2006

(51) Int. Cl. A61B 5/04

(52) U.S. Cl. 600/509

(57) ABSTRACT

A pre-conditioned ECG system and method includes a patient signaling device configured to alert the patient to minimize the patient's activity so that an ECG measurement may be taken, and further alerts the patient when the measurement is completed. The pre-conditioned ECG system and method also includes a pre-set buffer configured to hold a preset length of ECG data, and further configured to hold pre-set ECG parameters such as mean R-R interval, standard deviation of R-R interval, or simple rhythm analysis. The system and method may analyze the current 10 seconds of ECG data when the preset parameters are met or when selected parameters are met.

### FIELD OF THE INVENTION

The present invention is related to the field of cardiology. More particularly, the present invention is related to the field of specialized diagnostic cardiology.

### BACKGROUND OF THE INVENTION

An important aspect in the industry of diagnostic cardiology is correctly identifying and measuring the QT segment of an ECG signal correctly due to the physiological condition called QT prolongation. QT prolongation is a delay of normal heart beat cycle due recently to some prescription drugs. There is a lot of work being put into acquiring ECGs and clinical trials to measure QT prolongation and there is debate on whether Holter recordings for hours or regular ten second resting ECGs should be used.

Current electrocardiograph devices, or resting ECG devices acquire 10 second multi-lead readings, ranging from 12 to 16 leads simultaneously when the acquiring button is pushed on the device. This simple setting is adequate for some routine tasks, but is limited for other tasks, such as QT segment measurement, that need more accurate account of history data beyond the acquired 10 seconds. For example, during a clinical trial of a new pharmaceutical, what has become known as a Thorough QT Study (TQT) is performed. In the study, a series of ECGs are taken either by resting ECG devices or an ambulatory ECG device such as a Holter recorder, and heart rate corrected QT intervals are calculated from those ECGs.

When calculating a heart rate corrected QT interval, instantaneous heart rate based on 10 second ECG data might not be enough to reflect time-lag relationship of QT and RR interval, or “hysterisis.” Some studies suggest that an average heart rate based on 1-2 minutes prior ECG data is better than instantaneous heart rate for QT interval correction (QTc).

### SUMMARY OF THE INVENTION

A pre-conditioned ECG system and method includes a patient signaling device configured to alert the patient to minimize the patient's activity so that an ECG measurement may be taken, and further alerts the patient when the measurement is completed. The pre-conditioned ECG system and method also includes a pre-set buffer configured to hold a preset length of ECG data, and further configured to hold pre-set ECG parameters such as mean R-R interval, standard deviation of R-R interval, or simple rhythm analysis. The system and method may analyze the current 10 seconds of ECG data when the preset parameters are met or when selected parameters are met with preset criteria.

One aspect of the present disclosure is an ECG acquisition device for collecting ECG data from a patient, the ECG acquisition device comprising a set of ECG leads configured to connect to a patient and to collect a set of ECG data from the patient, a communication port configured to receive a set of instructional data, wherein the instructional data includes a set of start and stop times and a patient signaling mechanism configured to signal the patient when a start time occurs and when a stop time occurs, wherein the set of ECG data is collected from the patient between the start time and the stop time. The communications port is further configured with a signal output, wherein the signal output includes an audio signal corresponding to the start time and the stop time wherein either one of the communication port receiving the set of instructional data and sending the signal output is effectuated through a wired connection or the signal output is effectuated through a wireless connection. The set of ECG leads include at least one ECG lead or a standard lead set. The ECG acquisition device may be a Holter monitor. The patient signaling mechanism of the present disclosure includes any of the following: a light; an audible signal mechanism, wherein the audible signal mechanism is configured to produce any of an audible alarm or audible instructions and a vibrating mechanism. The ECG acquisition device further comprises of a data storage medium and a processor, the data storage medium configured to store the set of instructional data, and the processor configured to read and execute the set of instructional data.

Another aspect of the present disclosure is a method of collecting a set of ECG data from a patient, the method comprises connecting an acquisition device to the patient with a set of ECG leads, loading the acquisition device with a set of instructional data, wherein the set of instructional data includes a set of start times and stop times, alerting the patient when a start time arrives, wherein the start time signals that the set of ECG data is being recorded with the ECG leads, and alerting the patient when a stop time arrives, wherein the stop time signals that the recording of the ECG data has ended. The alerting step is effectuated by either a light, an audible signal mechanism, wherein the audible signal mechanism is configured to produce any of an audible alarm or audible instructions, and a vibrating mechanism. The loading step is effectuated by a communications port, wherein the communications port is further configured with a signal output, wherein the signal output includes an audio signal corresponding to the start time and the stop time. Further wherein either one of the loading step or the signal output is effectuated through either a wired connection or a wireless connection and wherein the set of ECG leads includes at least one ECG lead or a standard lead set and wherein the ECG acquisition device is a Holter monitor.

The present disclosure further includes a method of preconditioning an ECG system for collection of ECG data from a patient, the method comprises connecting an acquisition device to the patient with a set of ECG leads, loading the acquisition device with a set of pre-conditions, collecting and storing a prior segment of ECG data and a current segment of ECG data when the prior segment of ECG data satisfies the set of preconditions wherein the set of pre-conditions includes any of the following parameters mean R-R interval, standard deviation of R-R interval, variation of repolarization, wherein the variation of repolarization is a function of QT variation and T wave morphology variation and simple rhythm analysis. Further wherein a duration of the prior segment of ECG data and the current segment of ECG data is adjustable by a user. The method further comprises conducting the pre-determined ECG analysis of the prior segment of ECG data.

Yet another aspect of the present disclosure is a pre-condition ECG system, the system comprises a set of ECG leads configured to collect a prior segment of ECG data and a current segment of ECG data from a patient, a database for storing the prior and current segments of ECG data, a storage medium for storing a computer application and a set of pre-conditions, and a processing unit coupled to the set of ECG leads and the storage medium, and configured to execute the computer application, wherein when the computer application is executed, the current segment of ECG data is analyzed when the set of pre-conditions is satisfied, and further wherein a set of results are stored in the database.

### BRIEF DESCRIPTION OF THE DRAWINGS

FIG. 1 illustrates a block diagram illustrating a continuous ECG acquisition device with patient alert mechanism of an embodiment of the present invention.

FIG. 2 illustrates a flow chart according to an embodiment of the method of the present invention.

FIG. 3 illustrates a flow diagram of pre-conditioned ECG analysis method according to an embodiment of the present invention.

FIG. 4 illustrates a graphical representation of an unstable heart rate rhythm.

FIG. 5 illustrates a graphical representation of a normal, relatively stable heart rate rhythm.

FIG. 6 illustrates a block diagram according to an embodiment of the system of the present invention.

### DETAILED DESCRIPTION

The pre-conditioned ECG system and method includes a number of embodiments for avoiding inappropriate heart rate being used for calculating QTc value and other heart rate related correction values. One embodiment is a data acquisition device configured to alert a patient whose ECG is taken to keep as relaxed as possible prior to the data acquisition, so that his heart rate is in a stationary status when the ECG data is acquired. Another embodiment includes monitoring the heart rate 2-5 minutes prior and during the data acquisition, and storing the ECG with the most stationary status of heart rate. Another parameter can be the noise level of the signal. The data will be analyzed only when noise level is low, witch can be indicated with ‘Green’, ‘Yellow’, or ‘Red’ status. Here, ‘Green’ means signal is clean; ‘Yellow’ means signal is moderately noisy, and ‘Red’ means signal is very noisy. In one case, the signal will be stored and analyzed only when ‘Green’ status is shown. In another case, the signal can be stored and analyzed when either ‘Green’ or ‘Yellow’ status are shown. When ‘Red’ status is shown, patient will be reminded to relax, or stop moving or talking to improve the signal quality. The embodiment may be implemented separately or in combination.

In the second embodiment discussed above, the 10 seconds of ECG are preferably collected after or during certain events such as premature ventricular contraction (PVC), Paroxysmal Atrial fibrillation (PAFIB), T wave alternans (TWA), and other severe arrhythmia.

The data acquisition system of one embodiment generally includes an N-Lead Ambulatory ECG Recorder, where N is typically 12 or 3, but could be any number of leads>0, a patient signaling mechanism including any of the following: a speaker capable of playing back a recording; a simple audible alarm mechanism; a light; a buzzer or vibrating mechanism; a communications port for downloading the start/stop times, wherein the communications port could be a wired or wireless port; and/or an optional headphone jack to allow the user to wear headphones or a single ear piece to hear the signal.

In operation, a user downloads the start/stop times to the device. When a start time arrives, the recorder alerts the patient by playing a recording, e.g., “Please minimize activity”, buzzing, beeping, or turning the light on. The patient would than minimize physical activity until the recorder gave the stop signal which would be accomplished by playing another message, e.g., buzzing or turning the light off. It is contemplated the signal could be a combination of sound and light.

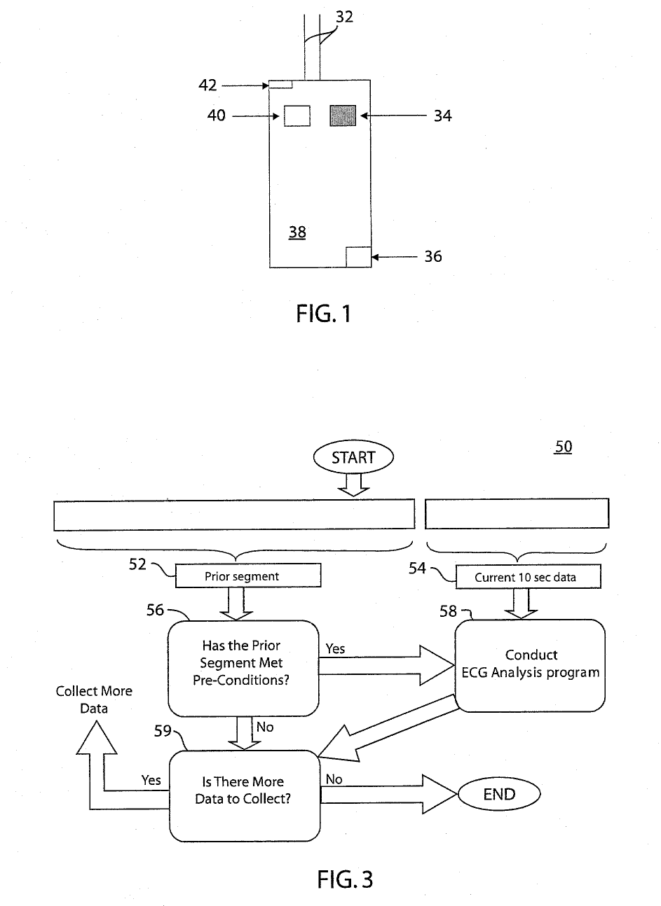

FIG. 1 illustrates an embodiment of the ECG acquisition device 30. Here, the ECG acquisition device 30 is illustrated as a Holter monitor 38, including a set of ECG leads 32 that connect to a patient, and a communications port 36. The communications port 36 may wirelessly or in a wired fashion, receive a set of data from the medical personnel administering this device, or from the clinical system monitoring this patient. In FIG. 1, the Holter recorder 38 also includes a headphone jack 42, that allows the patient to receive an audible signal from the Holter recorder 38 through a pair of headphones. It is contemplated however, that the communications port 36 may also include a headphone jack, or a wireless headphone device. The Holter recorder 38 also includes a speaker 40 and a light 34 for signaling start and stop times to the patient. In operation, the Holter recorder 38 is attached to the patient with the ECG leads 32, and a set of instructional data including the start and stop times is loaded through the communications port 36. The patient may utilize the headphone jack 42, or set the Holter recorder 38 for either an audible signal, visual signal, or both. When a start time arrives, the signal device that is selected will alert the patient that the Holter recorder 38 will begin recording ECG data. This can be done with a flashing light 34, a buzzer or audible instruction from the speaker 40, or the same through the headphone jack 42. This may also be effectuated by a vibrating mechanism (not shown). The noise level will be monitored in real-time. If the noise is above tolerance level, the patient will be reminded to relax further with either a visible or audible means. Clinician can overwrite the status to start the data collection. The Holter recorder 38 will then collect the ECG data through the ECG leads 32, and when a stop time arrives, the signaling devices will once again alert the patient. The collected ECG data may be communicated to medical personnel through the communications port 36 in a wireless or wired fashion, or the Holter recorder 38 may be collected by the medical personnel, and the data extracted in another matter. It should be noted once again that the ECG acquisition device 30 may be a Holter recorder 38 or any other applicable device in the medical field.

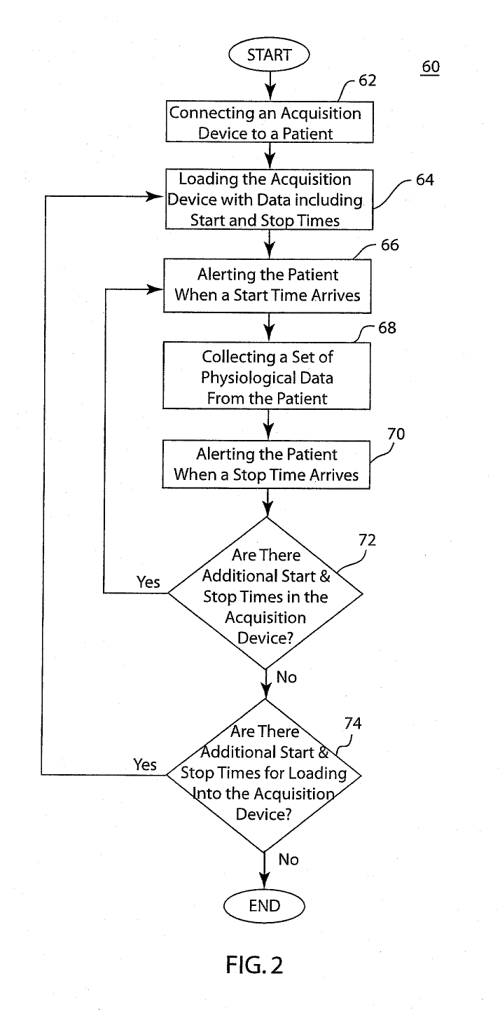

FIG. 2 includes an illustration of an embodiment of an acquisition method 60. In step 62, an acquisition device is connected to a patient. As stated previously, the acquisition device may be a Holter recorder or any other known device, and is connected to the patient through a set of ECG leads. In step 64, the acquisition device is loaded with data including start and stop times. In step 66, the patient is alerted when a stop time arrives, and in step 68, a set of physiological data including the ECG data is collected from the patient. In step 70, the patient is alerted when a stop time arrives, signaling the end of the ECG data collection. In step 72, if there are additional start and stop times in the acquisition device, then the method continues back to step 66. If there are no other additional start and stop times in the acquisition device, then the method continues on to step 74. In step 74, if there is more data to be loaded into the acquisition device, then the method continues on to step 64, and if no additional data exists, then the method ends. If there are no additional start and stop timer for loading into the acquisition device, the acquisition device may continue recording or may stop recording and be disconnected from the patient.

Another embodiment includes a pre-conditioned ECG system as shown in FIGS. 3-5. In this embodiment, a circular buffer holds a preset length of ECG data, e.g., 1-10 minutes depending on the setting. There are multiple parameter buffers: a current 10 second buffer, and a series of prior segment buffers. These buffers hold pre-set ECG parameters such as mean R-R interval, standard deviation of R-R interval, a variation of repolarization (T wave morphology change), or simple rhythm analysis.

One embodiment includes pre-condition options for the system such as analyzing the current 10 seconds of ECG data with an ECG analysis program regardless of the prior segment's condition with the aid of the device described above. Also, the system may analyze the current 10 seconds of ECG data only when a preset condition is met based on both the parameter buffers. For example, a similar mean and deviation of R-R intervals between the current 10 second data buffer and the prior data segments are reached, and/or the similar rhythm is reached such that HR prior≈Hrcurrent; and/or T wave morphology change is smaller than preset threshold. Furthermore, the system may analyze the current 10 seconds of ECG data unconditionally, but using selected parameters from the prior segment in the analysis. For example, using mean R-R interval of whole prior and current 10 second buffers for QTc calculation.

When the 10-second segment data buffer has reached a relatively stable condition, the analysis and calculation will be more accurate and reflect the underlying patient condition better. In the case of QT interval correction, it can reduce the hysterisis effect of certain parameters like RR/QT adaptation.

If waiting time exceeds preset time limit, the system and method may have the current 10 second data analyzed quickly but utilizing some parameters from prior segment. In some other cases, the pre-condition can be adjusted to wait for certain events to happen before analyzing current data segment.

FIG. 3 illustrates an embodiment on an analysis method 50. As described above, a prior segment step 52, and a current segment step 54 include collecting and recording an amount of ECG data from a patient wherein the prior segment of the data may be analyzed in order to decide when to use the current segment. Referring back to FIG. 3, in step 56 if the prior segment has met a set of pre-conditions, then the ECG analysis is conducted in step 58. If, in step 56 the prior segment has not met the pre-conditions, then in step 59 it is determined whether there is more data to collect. If there is more data to collect, then additional data is collected in steps 52 and 54. If there is no additional data to collect in step 59 then the method ends. Furthermore, in step 58, after the ECG analysis is conducted, the method moves onto step 59.

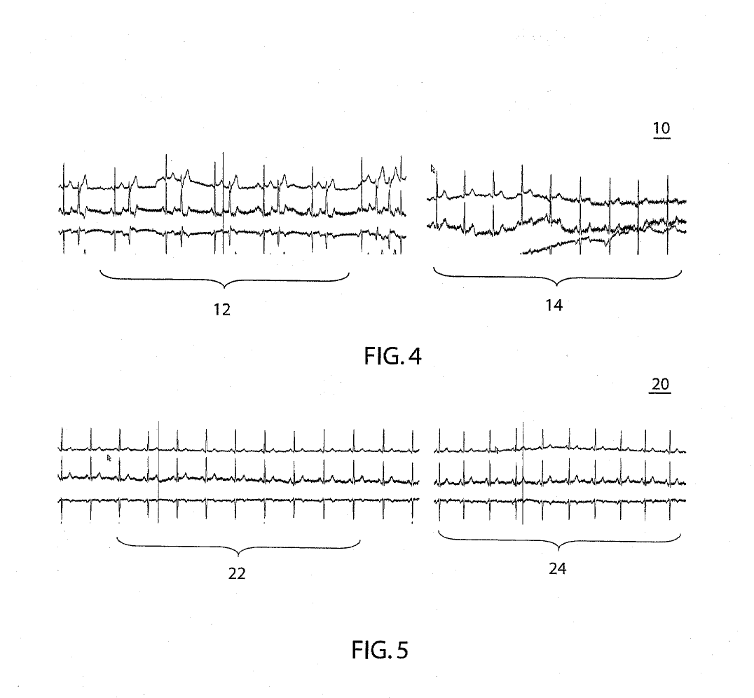

FIGS. 4 and 5 illustrate an unstable heart rhythm 10 compared to a stable heart rate rhythm 20. In the unstable heart rhythm, it is obvious that the unstable prior segment 12 produces an unstable current segment 14. Likewise, in the stable heart rate rhythm a stable prior segment 22 produces a stable current segment 24. FIGS. 4 and 5 are included in this discussion for illustrative purpose only. FIG. 4 illustrates how an unstable prior segment 12, that would probably not meet the preconditions of step 56 in FIG. 3, would produce the unstable current segment 14 as well. Likewise, the stable prior segment 22 would likely meet the prior segment preconditions set forth in step 56 of FIG. 3, thus allowing the system and method to conduct an ECG analysis on the stable current segment 24.

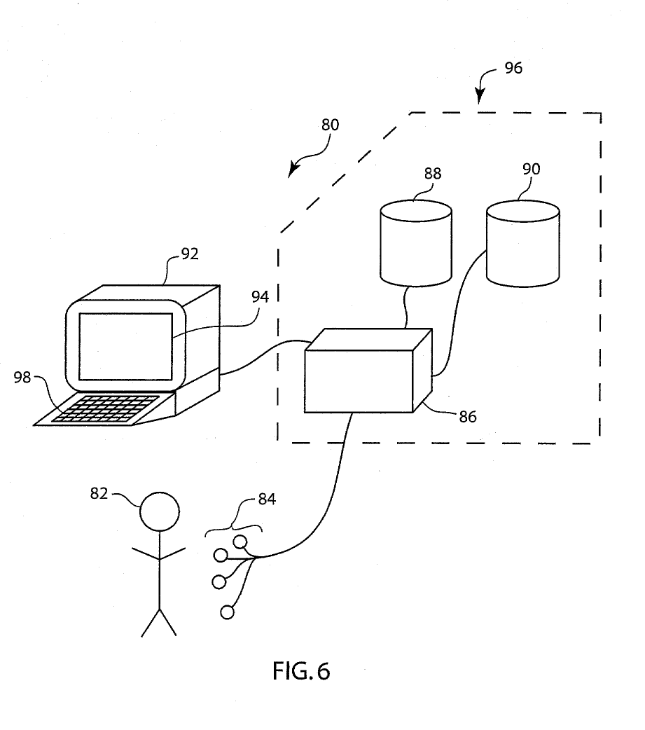

FIG. 6 illustrates an embodiment of an analysis system 80. The analysis system 80 includes a patient 82 being connected to a set of ECG leads 84, wherein the ECG leads 84 are coupled to the acquisition device 96. The acquisition device 96 includes a processing device 86, a storage medium 88 and a database 90. The acquisition device 96 may be coupled to an electronic device 92, including a graphical user interface (GUI) 94 and an input device 98. In operation, the acquisition device 96 receives a set of pre-conditions from a medical personnel, wherein such pre-conditions are loaded into the storage medium 88. The storage medium 88 also includes software capable of being executed on the processing device 86. The processing device 86 executes the software program, utilizing the loaded pre-conditions, and instructs the ECG leads 84 to collect and record a prior segment of ECG data and a current segment of ECG data from the patient 82 to the database 90. The software program conducts an ECG analysis on the segments stored in the database 90 where the pre-determined conditions are met. The segments and results are viewed by a user on the GUI 94 of the electronic device 92, and may manipulate the analysis with the results with the input device 98.

There are also several embodiments with respect to ECG data and parameter storage. A user may store only a current segment of ECG data ( e.g. 10 seconds segment) and pre-conditioned parameters. For example, the R-R interval series of prior segment. The user may also store both current and prior segments of ECG data and the parameters. This option will need more storage space, but with the advantage of reanalysis. Lastly, the user may store all current segment data and selected lead(s) of prior segment data.

The pre-condition technique can also be extended to continuous 12-lead Holter and stress ECG analysis when the individual segment of ECG analysis is used. The similar prior segment parameter and data buffers can be attached with current segment analysis.

The system and method of the present invention is advantageous as a more robust ECG parameter estimation, such as QTc used in pharma clinical trial. The quality of the recording of most clinically relevant data of the trial will be of the best quality possible under the circumstances. The system and method minimizes the time a human reader must spend with the analysis system looking for high quality 12-leads.

The present invention has been described in terms of specific embodiments incorporating details to facilitate the understanding of the principals of construction and operation of the invention. Such reference herein to specific embodiments and details thereof is not intended to limit the scope of the claims appended hereto. It will be apparent to those skilled in the art that modifications may be made in the embodiment chosen for illustration without departing from the spirit and scope of the invention.

What is claimed is:

1. An ECG acquisition device for collecting ECG data from a patient, the ECG acquisition device comprising:
a set of ECG leads configured to connect to a patient and to collect a set of ECG data from the patient;
a communication port configured to receive a set of instructional data, wherein the instructional data includes a set of start and stop times; and
a patient signaling mechanism configured to signal the patient when a start time occurs and when a stop time occurs,
wherein the set of ECG data is collected from the patient between the start time and the stop time.

2. The device as claimed in claim 1, wherein the patient signaling mechanism includes any of the following:
a light;
an audible signal mechanism, wherein the audible signal mechanism is configured to produce any of an audible alarm or audible instructions; and
a vibrating mechanism.

3. The device as claimed in claim 1, wherein the communications port is further configured with a signal output, wherein the signal output includes an audio signal corresponding to the start time and the stop time.

4. The device as claimed in claim 3, wherein either one of the communication port receiving the set of instructional data and sending the signal output is effectuated through a wired connection.

5. The device as claimed in claim 3, wherein either one of the communication port receiving the set of instructional data and sending the signal output is effectuated through a wireless connection.

6. The device as claimed in claim 1, wherein the set of ECG leads include at least one ECG lead.

7. The device as claimed in claim 1, wherein the set of ECG leads includes a standard lead set.

8. The device as claimed in claim 1, wherein the ECG acquisition device is a Hotter monitor.

9. The device as claimed in claim 1, further comprising a data storage medium and a processor, the data storage medium configured to store the set of instructional data, and the processor configured to read and execute the set of instructional data.

10. A method of collecting a set of ECG data from a patient, the method comprising:
connecting an acquisition device to the patient with a set of ECG leads;
loading the acquisition device with a set of instructional data, wherein the set of instructional data includes a set of start times and stop times;
alerting the patient when a start time arrives, wherein the start time signals that the set of ECG data is being recorded with the ECG leads; and
alerting the patient when a stop time arrives, wherein the stop time signals that the recording of the ECG data has ended.

11. The device as claimed in claim 10, wherein the alerting step is effectuated by any of the following:
a light;
an audible signal mechanism, wherein the audible signal mechanism is configured to produce any of an audible alarm or audible instructions; and
a vibrating mechanism.

12. The method as claimed in claim 10, wherein the loading step is effectuated by a communications port, wherein the communications port is further configured with a signal output, wherein the signal output includes an audio signal corresponding to the start time and the stop time.

13. The method as claimed in claim 12, wherein either one of the loading step or the signal output is effectuated through a wired connection.

14. The method as claimed in claim 12, wherein either one of the loading step or the signal output is effectuated through a wireless connection.

15. The method as claimed in claim 10, wherein the set of ECG leads include at least one ECG lead.

16. The method as claimed in claim 10, wherein the set of ECG leads includes a standard lead set.

17. The method as claimed in claim 10, wherein the ECG acquisition device is a hotter monitor.

18. A method of preconditioning an ECG system for collection of ECG data from a patient, the method comprising:
connecting an acquisition device to the patient with a set of ECG leads;
loading the acquisition device with a set of pre-conditions;
collecting and storing a prior segment of ECG data and a current segment of ECG data when the prior segment of ECG data satisfies the set of preconditions.

19. The method as claimed in 18, wherein the set of pre-conditions includes any of the following parameters:
mean R-R interval;
standard deviation of R-R interval;
variation of repolarization, wherein the variation of repolarization is a function of QT variation and T wave morphology variation; and
simple rhythm analysis.

20. The method as claimed in claim 18, wherein a duration of the prior segment of ECG data and the current segment of ECG data is adjustable by a user.

21. The method as claimed in claim 18, further comprising conducting the pre-determined ECG analysis of the prior segment of ECG data.

22. A pre-condition ECG system, the system comprising:
a set of ECG leads configured to collect a prior segment of ECG data and a current segment of ECG data from a patient;
a database for storing the prior and current segments of ECG data;
a storage medium for storing a computer application and a set of pre-conditions; and
a processing unit coupled to the set of ECG leads and the storage medium, and configured to execute the computer application,
wherein when the computer application is executed, the current segment of ECG data is analyzed when the set of pre-conditions is satisfied, and further wherein a set of results are stored in the database.
